(19) United States
(12) Patent Application Publication
(10) Pub. No.: US 2023/0415145 A1
(43) Pub. Date: Dec. 28, 2023

(54) DEVICES AND METHODS FOR SAMPLE COLLECTION

(71) Applicant: Predicine, Inc., Hayward, CA (US)

(72) Inventors: Shujun LUO, Hayward, CA (US); Pan DU, Dublin, CA (US); Shidong JIA, Hayward, CA (US)

(21) Appl. No.: 18/340,562

(22) Filed: Jun. 23, 2023

Related U.S. Application Data
(63) Continuation of application No. PCT/US23/25958, filed on Jun. 22, 2023.
(60) Provisional application No. 63/354,883, filed on Jun. 23, 2022.

(51) Int. Cl. B01L 3/00

(52) U.S. Cl. CPC B01L 3/502; B01L 2200/0689; B01L 2300/042; B01L 2300/0672

(57) ABSTRACT

Provided herein are devices and methods for collection of a biological sample. The device may comprise a cap, a pierceable container comprising a reagent for processing or preserving a biological sample, and a container that comprises a piercer. Upon engagement of the cap with an opening of the container, the piercer may be able to pierce a pierceable container, thereby releasing the reagent of the pierceable container into the container. The methods may comprise use of the device to collect a biological sample. The methods may comprise assaying analytes of the biological sample.

### CROSS-REFERENCE

This application is a continuation of International Application No. PCT/US23/25958, filed Jun. 22, 2023, which claims the benefit of U.S. Provisional Application No. 63/354,883, filed Jun. 23, 2022, each of which is incorporated by reference herein in its entirety.

### BACKGROUND

Disease detection is a critical component for providing treatment for patients, and early detection may improve outcomes. Collecting biological samples from a subject (e.g., patient) may allow for disease detection to be performed on the biological fluids or tissues of a subject. Sample collection may occur at a time or location that is distinct from the location that an analysis of the sample is performed. A sample may need to maintain its integrity prior to analysis in order to provide an accurate actionable data.

### SUMMARY

Provided herein are devices and methods for collection of a biological sample from a subject. Biological samples collected from a subject may comprise nucleic acids, polypeptides, or other biomolecules or analytes. The presence of these biomolecules or analytes may be indicative of a genotypic state, phenotypic state, or other characteristic of a subject. However, upon collection and prior to analysis of a sample, biomolecules or analytes may undergo degradation or other reactions, such that the collected sample and the analyzed sample may comprise discrepancies. Thus there is a need for sample collection devices and methods that may improve the preservation of biomolecules or analytes in a sample. The devices and methods provided herein may be suitable for collection and preservation of a sample from a subject.

In an aspect, the present disclosure provides a device for collecting a biological sample of a subject, comprising: a container comprising a piercer extending across a longitudinal axis of the container towards an opening of the container; and a cap comprising a pierceable container comprising a reagent for processing or preserving the biological sample, wherein the device is configured such that upon engagement of the cap with the container to dispose the cap adjacent to the opening, the piercer pierces the pierceable container to release the reagent into the container.

In some embodiments, the piercer pierces the pierceable container upon rotation of the cap or the container.

In some embodiments, the engagement of the cap with the container to dispose the cap adjacent to the opening, and movement of the cap towards the container or movement of the container towards the cap, closes the container.

In some embodiments, the cap is configured to be attached to the container. In some embodiments, the cap is configured to be attached to the container at least in part by exerting a pressure on the cap towards the container or exerting a pressure on the container towards the cap, when the cap is adjacent to the opening. In some embodiments, the cap is configured to be attached to the container at least in part by rotating the cap or the container, when the cap is adjacent to the opening. In some embodiments, the attaching of the cap to the container generates a leak-resistant, leak-proof, or airtight compartment.

In some embodiments, the cap and the container each comprises threading, wherein the threading of the cap and the threading of the container are configured to interface together thereby closing the container.

In some embodiments, the piercer comprises a rod, wherein a top of the rod comprises a sharp protrusion. In some embodiments, the piercer comprises a plurality of sharp protrusions. In some embodiments, the rod is disposed on a surface of the container. In some embodiments, the rod is disposed on a bottom surface of the container.

In some embodiments, the pierceable container is configured such that a pierceable surface of the pierceable container is exposed on a bottom surface of the cap.

In some embodiments, the cap comprises a solid surface at a top surface and at side surfaces of the cap.

In some embodiments, the reagent is configured to preserve integrity of analytes in the biological sample, responsive to the releasing of the reagent into the container. In some embodiments, the analytes comprise nucleic acids, polypeptides, lipids, or carbohydrates.

In some embodiments, the sharp protrusion is located above a plane of the opening. In some embodiments, the sharp protrusion is configured to protrude into the container.

In some embodiments, the container comprises a tube. In some embodiments, the container comprises a flat bottom or a substantially flat bottom. In some embodiments, the container comprises a round bottom or a substantially round bottom.

In some embodiments, the pierceable container comprises a pouch.

In some embodiments, the reagent comprises an enzyme, an enzyme inhibitor, a chelator, a buffering agent, or a combination thereof. In some embodiments, the chelator comprises ethylenediaminetetraacetic acid (EDTA).

In some embodiments, the biological sample comprises a urine sample.

In another aspect, the present disclosure provides a method for collecting a biological sample of a subject, comprising: (a) providing a container comprising a piercer extending across a longitudinal axis of the container towards an opening of the container, wherein the container comprises the biological sample; (b) engaging a cap with the container to dispose the cap adjacent to the opening, wherein the cap comprises a pierceable container comprising a reagent for processing or preserving the biological sample; and (c) upon engaging the cap with the container, subjecting the cap or the container to a closing motion, thereby piercing, by the piercer, the pierceable container to release the reagent into the container.

In some embodiments, the closing motion comprises rotating the container or the cap.

In some embodiments, the closing motion comprises exerting a pressure on the cap towards the container or exerting a pressure on the container towards the cap.

In some embodiments, the method further comprises closing the container at least in part by rotating the cap or the container.

In some embodiments, the method further comprises closing the container at least in part by exerting a pressure on the cap or the container.

In some embodiments, the method further comprises attaching the cap to the container.

In some embodiments, the attaching of the cap to the container generates a leak-resistant, leak-proof, or airtight compartment.

In some embodiments, the cap and the container each comprise threading, and the method further comprises interfacing together the threading of the cap and the threading of the container thereby closing the container.

In some embodiments, the piercer comprises a rod, wherein a top of the rod comprises a sharp protrusion. In some embodiments, the piercer comprises a plurality of sharp protrusions. In some embodiments, the rod is disposed on a surface of the container. In some embodiments, the rod is disposed on a bottom surface of the container.

In some embodiments, the pierceable container is configured such that a pierceable surface of the pierceable container is exposed on a bottom surface of the cap.

In some embodiments, the cap comprises a solid surface at a top surface and at side surfaces of the cap.

In some embodiments, the method further comprises mixing together the reagent and the biological sample, responsive to the releasing of the reagent into the container. In some embodiments, the reagent is configured to preserve integrity of analytes in the biological sample, responsive to the mixing.

In some embodiments, the analytes comprise nucleic acids, polypeptides, lipids, or carbohydrates. In some embodiments, the method further comprises assaying the analytes.

In some embodiments, the sharp protrusion is located above a plane of the container opening. In some embodiments, the sharp protrusion protrudes into the container.

In some embodiments, the container comprises a tube. In some embodiments, the container comprises a flat bottom or a substantially flat bottom. In some embodiments, the container comprises a round bottom or a substantially round bottom. In some embodiments, the pierceable container comprises a pouch.

In some embodiments, the reagent comprises an enzyme, an enzyme inhibitor, a chelator, a buffering agent, or a combination thereof. In some embodiments, the chelator comprises ethylenediaminetetraacetic acid (EDTA).

In some embodiments, the method further comprises, prior to (a), providing the biological sample to the container.

In some embodiments, the subject has or is suspected of having a disease, a disorder, a genetic disorder, or a genetic aberration.

In some embodiments, the disease comprises cancer. In some embodiments, the cancer comprises a genitourinary cancer. In some embodiments, the genitourinary cancer comprises a bladder cancer, a prostate cancer, a kidney or renal cancer, a penile cancer, a testicular cancer, or a urethral cancer.

In some embodiments, the biological sample comprises a urine sample.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-1B show an example of a device for collection of a biological sample, including a view of an open configuration (FIG. 1A) and a view of a closed configuration (FIG. 1B).

FIGS. 2A-2F show an example of a device for collection of a biological sample, including example dimensions of the device (FIG. 2A), a view of a container (FIG. 2B), and various views of a cap (FIGS. 2C-2F).

FIGS. 3A-3C show an example of a device for collection of a biological sample, including a side view (FIG. 3A), a cross-sectional side view (FIG. 3B), and an oblique view (FIG. 3C).

FIGS. 4A-4D show an example of a cap for use in a device for collection of a biological sample, including a side view of the cap (FIG. 4A), a cross-sectional side view of the cap inside a closed container (FIG. 4B), a top view of the cap (FIG. 4C), and an oblique view of the cap (FIG. 4D).

FIGS. 5A-5E show an example of a container for use in a device for collection of a biological sample, including a side view (FIG. 5A), a cross-sectional side view (FIG. 5B), a bottom view (FIG. 5C), a top view (FIG. 5D), and an oblique view (FIG. 5E).

FIGS. 6A-6C show an example of a device for collection of a biological sample, including a view of an open configuration (FIG. 6A), a view of a closed configuration (FIG. 6B), and a top view of a container in an open configuration (FIG. 6C).

FIG. 7 shows a computer system 701 that is programmed or otherwise configured to implement a method of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject” generally refers to an entity or a medium that has testable or detectable information. A subject can be a person, individual, or patient. A subject can be a vertebrate, such as, for example, a mammal. Non-limiting examples of mammals include humans, simians, farm animals, sport animals, rodents, and pets. The subject can be a person that has a disease, disorder, or condition (e.g., cancer) or is suspected of having a disease, disorder, or condition (e.g., cancer). The subject may be displaying a symptom indicative of a health, physiological state, or condition of the subject, such as a cancer or other disease, disorder, or condition of the subject. As an alternative, the subject can be asymptomatic with respect to such health or physiological state or condition.

As used herein, the term “sample” generally refers to a biological sample obtained from or derived from one or more subjects. Biological samples may be cell-free biological samples or substantially cell-free biological samples or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), proteins, antibodies, plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck® RNA Complete BCT®), or a cell-free DNA collection tube (e.g., Streck ° Cell-Free DNA BCT®). Cell-free biological samples may be derived from whole blood samples by fractionation (e.g., by differential centrifugation). Biological samples or derivatives thereof may contain cells. For example, a biological sample may be a blood sample or a derivative thereof (e.g., blood collected by a collection tube or blood drops).

As used herein, the term “cell-free sample” generally refers to a biological sample that is substantially devoid of intact cells. A cell-free sample may be derived from a biological sample that is itself substantially devoid of cells or may be derived from a sample from which cells have been removed. Non-limiting examples of cell-free samples include those derived from blood, serum, plasma, urine, semen, sputum, feces, ductal exudate, lymph, and recovered lavage.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

Disease detection is a critical component for providing treatment for patients, and early detection may improve outcomes. Collecting biological samples from a subject (e.g., patient) may allow for disease detection to be performed on the biological fluids or tissues of a subject. Sample collection may occur at a time or location that is distinct from the location that an analysis of the sample is performed. A sample may need to maintain its integrity prior to analysis in order to provide an accurate actionable data.

Provided herein are devices and methods for collection of a biological sample from a subject. Biological samples collected from a subject may comprise nucleic acids, polypeptides, or other biomolecules or analytes. The presence of these biomolecules or analytes may be indicative of a genotypic state, phenotypic state, or other characteristic of a subject. However, upon collection and prior to analysis of a sample, biomolecules or analytes may undergo degradation or other reactions, such that the collected sample and the analyzed sample may comprise discrepancies. Thus there is a need for sample collection devices and methods that may improve the preservation of biomolecules or analytes in a sample. The devices and methods provided herein may be suitable for collection and preservation of a sample from a subject.

The devices and methods provided herein may be used to collect and preserve a sample to allow for the transport of preserved samples to an analysis location. The sample may then be processed such that one or more types of analytes may be analyzed. The one or more types of analytes may comprise DNA or RNA, for example cfDNA or cfRNA. The one or more analytes may be cfDNA, germline DNA, and cfRNA. The one or more types of analytes may comprise proteins. The one or more types of analytes may comprise metabolites. The devices and methods may allow for improved detection or determination of a diagnosis or prognosis of a subject, as compared to a sample that is not subjected to the devices and methods of this disclosure.

Provided herein is a device for collecting a urine sample of a subject, comprising: a container comprising a piercer extending across a longitudinal axis of the container towards an opening of the container; and a cap comprising a pierceable container comprising a reagent for processing or preserving the urine sample, wherein the device is configured such that upon engagement of the container with the cap to dispose the cap adjacent to the opening, the piercer pierces the pierceable container to release the reagent into the container.

Provided herein is a method for collecting a urine sample of a subject, comprising: (a) providing a container comprising a piercer extending across a longitudinal axis of the container towards an opening of the container, wherein the container comprises the urine sample; (b) engaging the container with a cap such that the cap is disposed adjacent to the opening, wherein the cap comprises a pierceable container comprising a reagent for processing or preserving the urine sample; and (c) upon engagement of the container with the cap, subjecting the cap or the container to a closing motion, which closing motion causes the piercer to pierce the pierceable container to release the reagent into the container.

In some embodiments, the device or method comprises a container. The container may be configured to hold a sample that has been added to the container. The container may comprise a tube. The container may comprise a rounded bottom. The container may comprise a flat bottom. The container may comprise a bottom with small ridges. The small ridges may allow the container to stably rest on a flat surface or may allow an instrument to interface with the container. A piercer may be directly affixed to the container. The container may comprise a round opening. The container may comprise a surface for affixing a label, or may comprise a surface that can be written or drawn on. For example, the container may comprise a matte surface. The matte surface may allow pen marks or other marking to be visible on a container which would be less visible (or less legible) on a transparent container. The label may be a separate component, for example, a sticker that can be added to the container, or the label may be printed, engraved, etched, or otherwise added directly to the container. For example, the container may have a location for filling in a name of a subject from which the sample is derived. Similarly, the container may have a location for indicating a room number, doctors name, or other information regarding a medical provider. Additionally, the container may comprise a indicated location for adding in information relating to a date and time in which the sample was taken. This may allow for the sample to be tracked as it is distributed to other individuals for processing. For example, date and time information may allow a medical provider to track when the sample was provided.

The container may comprise a threading to interface with a cap and allow a cap to be screwed on to the container. The threading may be a single threading. The threading may be a double threading. The threading on a container may be spaced, have a width, or thread pitch such to interface with threading on a cap.

The volume of the container may be at least 10 mL, 15 mL, 20 mL, 25 mL, 30 mL, 40 mL, 50 mL, 55 mL, 60 mL, 65 mL, 70 mL, 75 mL, 80 mL, 85 mL, 90 mL, 95 mL, 100 mL, 110 mL, 120 mL, 130 mL, 140 mL, 150 mL, 160 mL, 170 mL, 180 mL, 190 mL, 200 mL, or more. The volume of the container may be no more than 10 mL, 15 mL, 20 mL, 25 mL, 30 mL, 40 mL, 50 mL, 55 mL, 60 mL, 65 mL, 70 mL, 75 mL, 80 mL, 85 mL, 90 mL, 95 mL, 100 mL, 110 mL, 120 mL, 130 mL, 140 mL, 150 mL, 160 mL, 170 mL, 180 mL, 190 mL, 200 mL, or less.

In some embodiments, the device or method comprises a piercer. The piercer may comprise a piercing rod. The piercer may comprise a sharp object that is configured to pierce a pierceable container. The sharp object may be arranged at various orientations in relation to a piercing rod. For example, a piercing rod may be arranged along a vertical axis, and the sharp object may be affixed directly on top of the piercing rod with a sharp edge in line with the vertical axis. For example, a piercing rod may be arranged along a vertical axis, and the sharp object may be affixed directly on top of the piercing rod with a sharp edge at an angle from the vertical axis. The piercer may comprise a plurality of sharp objects. The sharp objects may comprise a sharp edge along a vertical axis or at an angle to the vertical axis. The piercer may be affixed to a bottom surface of the container or another surface of the container, and may extend to different planes toward the opening of the container. The piercer may extend to the plane of the opening of the container. The piercer may extend past a plane of the opening of the container.

In some embodiments, the device or method comprises a cap. The cap may comprise a round cap. The cap may be configured to hold or comprise a pierceable container. For example, the cap may comprise a compartment that protrudes perpendicularly into the container. This additional compartment can hold a pierceable container. The volume of the compartment may be at least 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, or more. The volume of the compartment may be no more than 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, or less.

The bottom of the cap may allow for the pierceable container to be exposed (e.g., such that a pierceable surface of the pierceable container is exposed) and may be pierced by a piercer The cap may be allow for attachment to the container. For example, the cap may comprise threading and corresponding threading may be present on the container to allow for the cap to be rotated or screwed onto the container thereby closing the container (e.g., to provide a leak-resistant, leak-proof, and/or airtight seal). The cap may comprise a single thread. The cap may comprise a double thread. The threading on a cap may be spaced, have a width, or a thread pitch such to interface with threading on a container. The cap may comprise a gasket to allow for a leak proof container to be generated once the cap is attached to the container. The cap may be attached to the container upon exerting pressure on the cap towards the container. The cap may be configured to close multiple different types of containers.

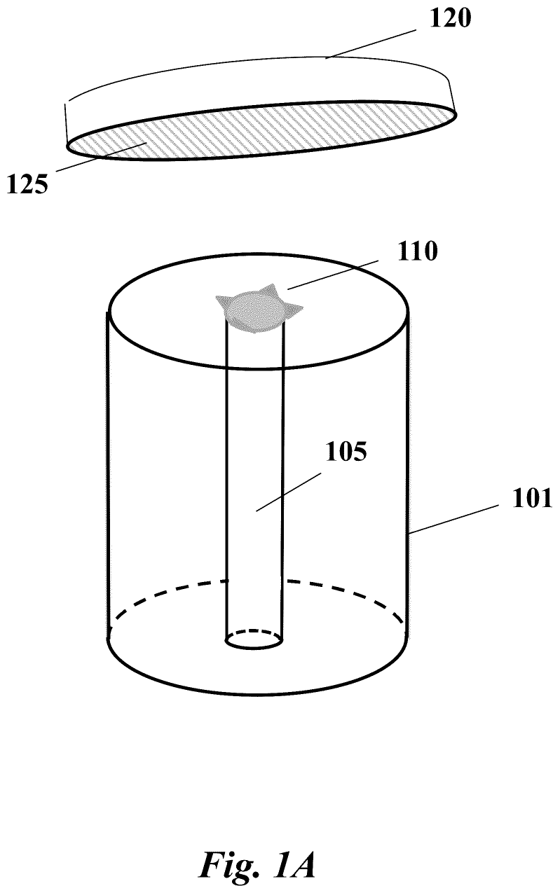

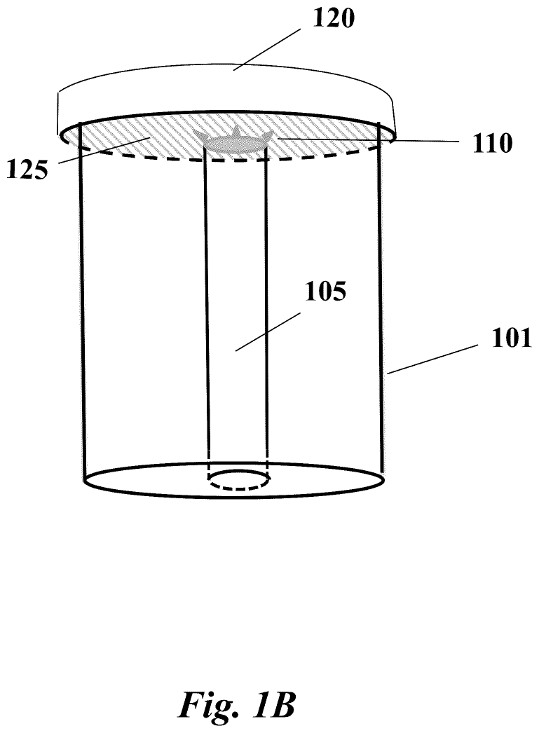

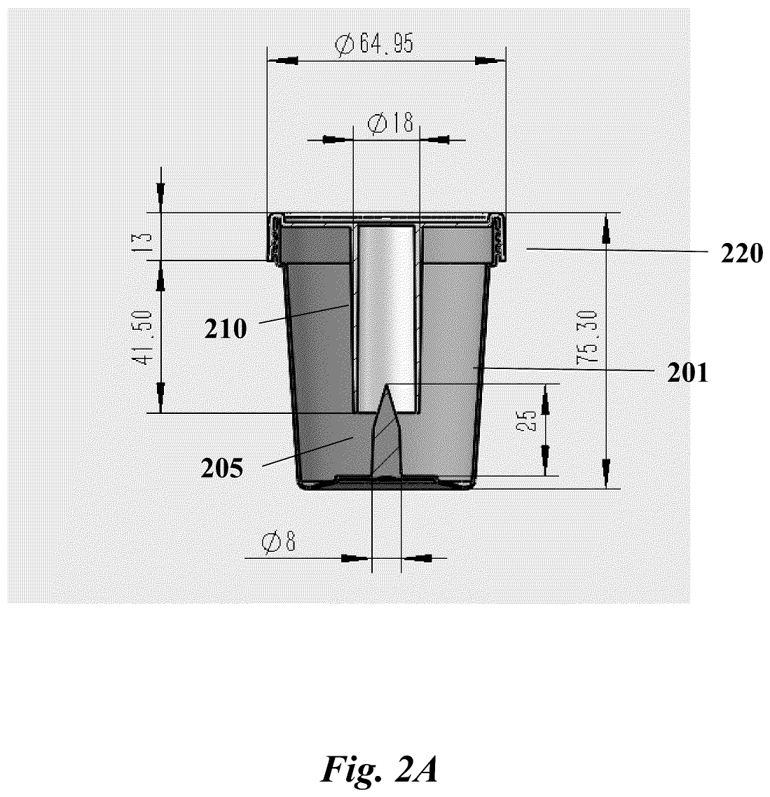

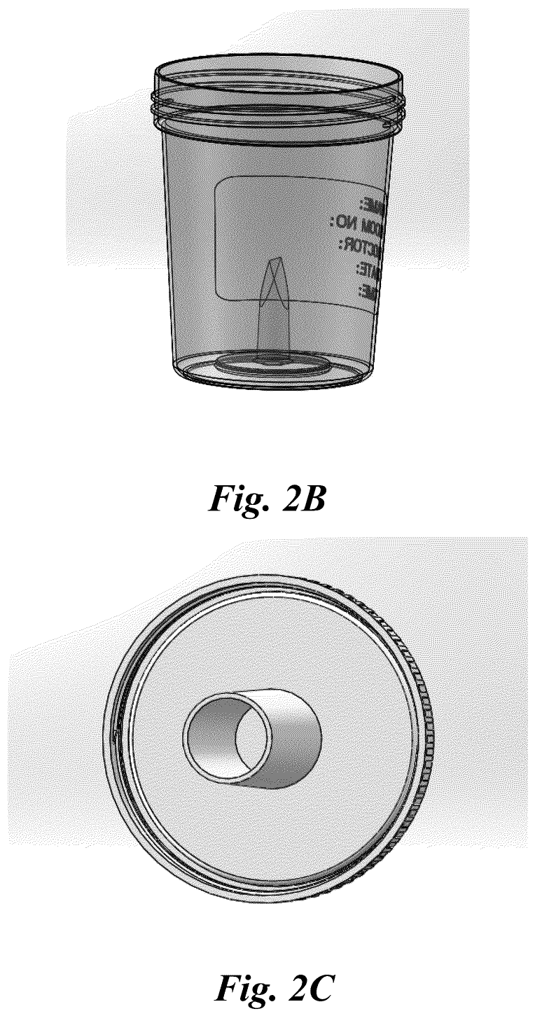

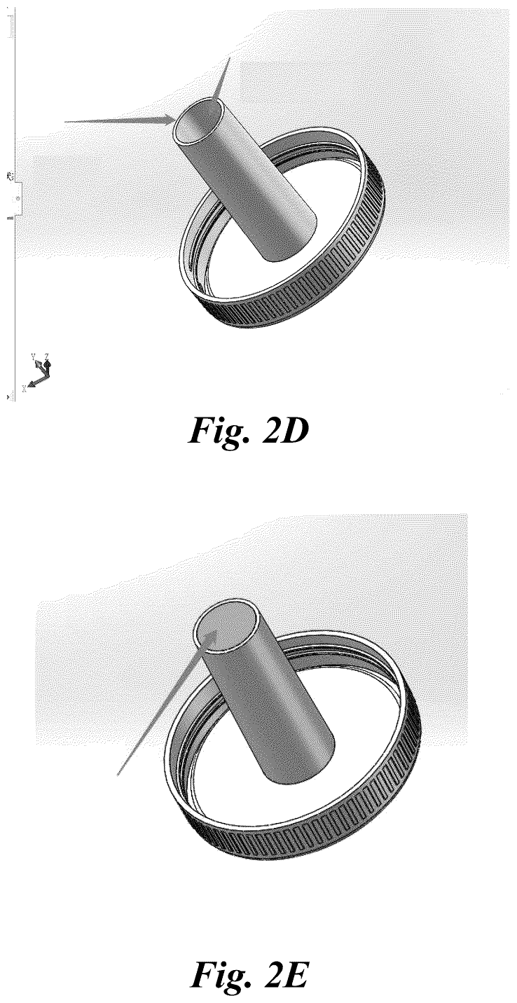

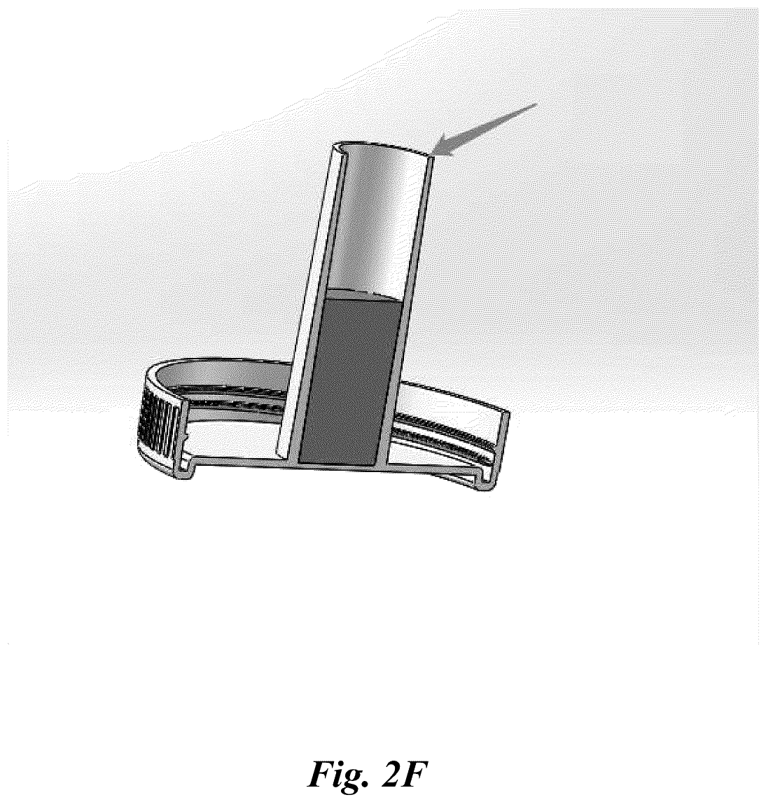

In some embodiments, the device or method comprises a pierceable container. The pierceable container may be a compartment attached to the cap with a closure such a membrane, seal, or cap; or the pierceable container may be an adapter attached to the cap where a bottle or pouch may fit into it. The pierceable container may have different sizes in diameter and different protruding lengths into the container. For example, the pierceable container may have a diameter maximal and protruding length minimal, as shown in FIG. 1A. As another example, the pierceable container may have a diameter intermediate and protruding length intermediate, as shown in FIG. 2C. The pierceable container may comprise a flexible material. The pierceable container may comprise a pierceable surface that can be pierced such that liquid inside the pierceable container may leave the pierceable container. The pierceable container may comprise a plurality of pierceable surfaces. The pierceable container may comprise only one pierceable surface.

The pierceable container may comprise a volume of at least 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, or more. The pierceable container may comprise a volume of no more than 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, or less.

FIG. 1A-1B show an example of a device for collection of a biological sample, including a view of an open configuration (FIG. 1A) and a view of a closed configuration (FIG. 1B).

FIG. 1A shows an example of the device for collection of a biological sample in an open configuration. A container 101 may comprise a piercing rod 105, which is affixed to a bottom surface of the container and extends along a vertical axis. At the top of the piercing rod 105, a piercer 110 is attached. The piercer 110 may comprise one or multiple sharp object or edges. These edges may extend past the plane or the opening of the container 101. The device may comprise a cap 120, which may be used to close the container 101. The cap may comprise a pierceable container 125, which is attached to or is part of the cap 120. The pierceable container may be exposed at the bottom of the cap such that when the cap 120 is brought to the opening of the container 101, the pierceable container 125 is adjacent to the piercer 110. Upon attachment of the cap 120 to the container 101, the piercer 110 makes contact with the pierceable container 125, and pierces through the pierceable surface of the pierceable container 125. Upon piercing of the pierceable container, the contents of the pierceable container may be able to exit the pierceable container. With the aid of gravity, the contents of the pierceable container may exit the pierceable container 125 and enter the container 101, and mix with the existing contents of the container (e.g., a urine sample). The closing of the container 101 with the cap 120 may therefore generate a leak-resistant, leak-proof, airtight, or otherwise stable container comprising a sample and simultaneously allow for the addition of a reagent that may help preserve or stabilize the sample in the container.

FIG. 1B shows an example of the device for collection of a biological sample in a closed configuration. The cap 120 has been attached to the container 101. The piercer 110 is now in direct contact with the pierceable container 125, and pierces the pierceable container 125. When the container is closed, the contents of the pierceable container 125 exit the pierceable container 125 and flow into closed container 101. The contents in the container 101 can now mix with the existing contents in the pierceable container, allowing for the introduction of reagents into the container 101 while simultaneously closing the container.

FIG. 2A-2F show an example of a device for collection of a biological sample, including example dimensions of the device (FIG. 2A), a view of a container (FIG. 2B), and various views of a cap (FIGS. 2C-2F). A container 201 comprising a piercing rod/piercer 205 is closed with a cap 220 comprising a pierceable container 210. The piercer 205 extends into the pierceable container 210 and allows the release of any reagent into the container.

FIG. 2A shows example dimensions of the device. FIG. 2B shows an alternative angle of container 201. Container 201 shows with threading on the upper portion which allows for interfacing with the cap 210 and allows for the cap to screw on to the container. Cap 210 has threading in order to allow the container to be closed. The piercer is affixed to the inside bottom surface of the container and has a single pointed end that is able to pierce a pierceable container. The container may be clear such to allow fluids in the container to be visualized. This may allow for a user to identify the container as containing fluids. Additionally, a color of a fluid may be indicative of a type of fluid or presence of a reagent. The container may also comprise words or marking that allow for the identification of the sample, or of a particular patient or user from which the sample is derived.

FIG. 2C shows an alternate angle of the cap 210. The cap 210 comprises a threading to allow for interfacing with the container such that the container may be closed. Additional cap 210 comprises an open space to allow for the insertion of a pierceable container. The pierceable container may be interchanged into a cap and allow for a modular design wherein a generic cap may be used and different pierceable containers may be added into the cap based on a desired application. In some embodiments, the cap 210 can be used as a pierceable container directly, with a pierceable membrane or seal or cap to close the pink opening.

FIG. 2D shows another angle of the cap 210. As described above, the cap 210 comprises a cylinder that is hollow that can be filled with the pierceable container. The outside of the cylinder can be made of the same material as the cap, and may be a hard that is not pierceable by a piercer or piercing rod. When properly oriented, the outside of the cylinder should not contact the piercer and instead should envelop the piercer such that it contacts an object that is placed in the opening that is generated by the cylinder.

FIG. 2E shows a similar angle as FIG. 2D, with the cylinder or other shapes, filled with the pierceable container or reagent. This cap 210 can now be used to add reagent to closed container upon the engagement of the cap to the container. FIG. 2F shows a cross section the cap. The cylinder may have a solid wall surrounding the reagent/pierceable container with only one open end.

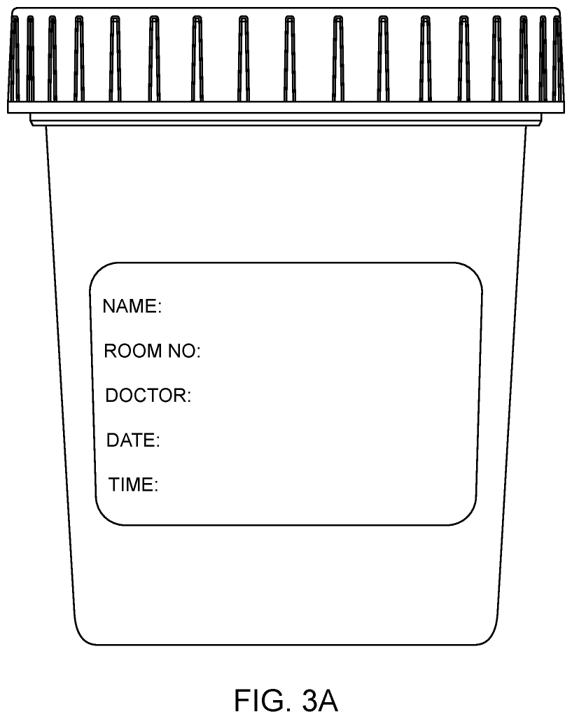

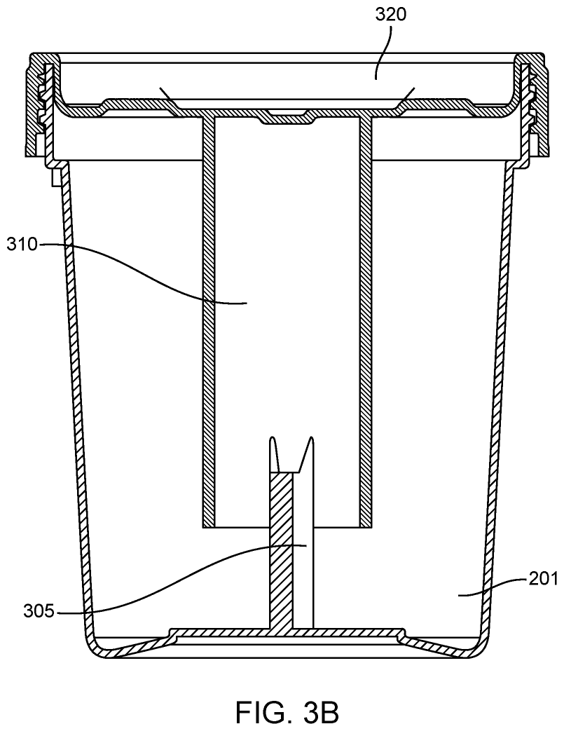

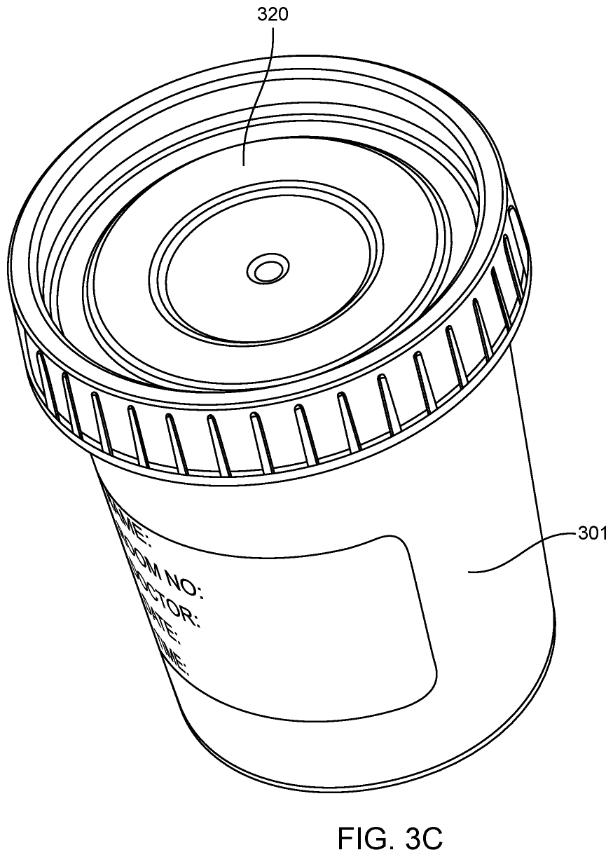

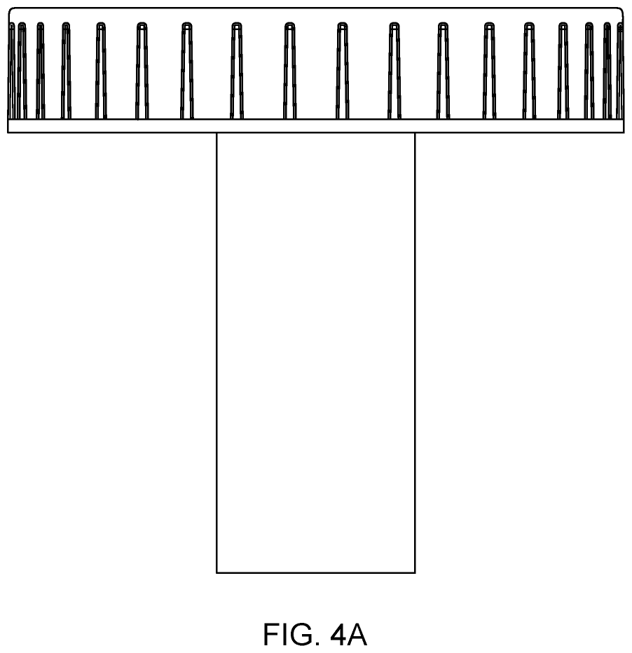

FIGS. 3A-3C show an example of a device for collection of a biological sample, including a side view (FIG. 3A), a cross-sectional side view (FIG. 3B), and an oblique view (FIG. 3C).

FIG. 3A shows an example exterior of the collection device. As shown, the device may comprise a label or location of the device for writing or adding information pertaining to the sample. As shown, the device may have a location for filling in a name of a subject from which the sample is derived. Similarly, the device may have a location for indicating a room number, doctors name, or other information regarding a medical provider. Additionally, the device may comprise a indicated location for adding in information relating to a date and time in which the sample was taken.

FIG. 3B shows a cross section view of an example device with example dimensions. A container 301 comprising a piercing rod/piercer 305 is closed with a cap 320 comprising a pierceable container 310. The piercer 305 extends into the pierceable container 310 and allows the release of any reagent into the container. The piercer depicted in FIG. 3B a multi-pronged piercer as opposed to a single pronged piercer depicted in FIG. 2A. FIG. 3C shows an oblique view of the closed device with cap 320 and container 301.

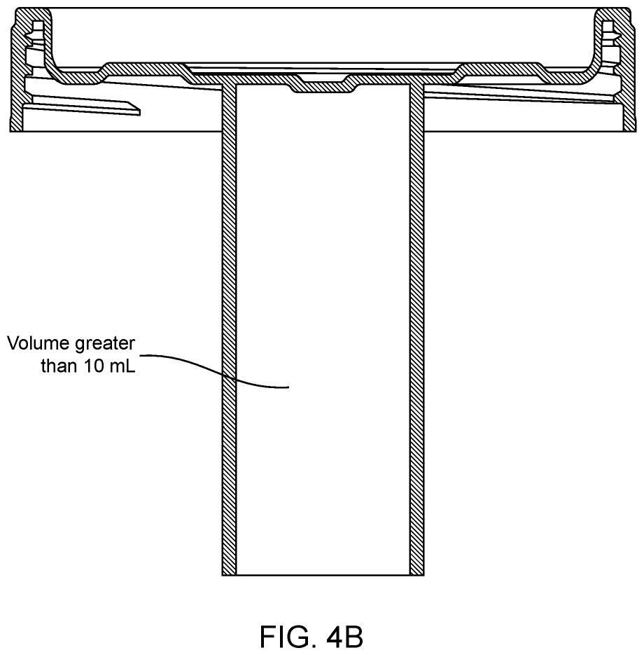

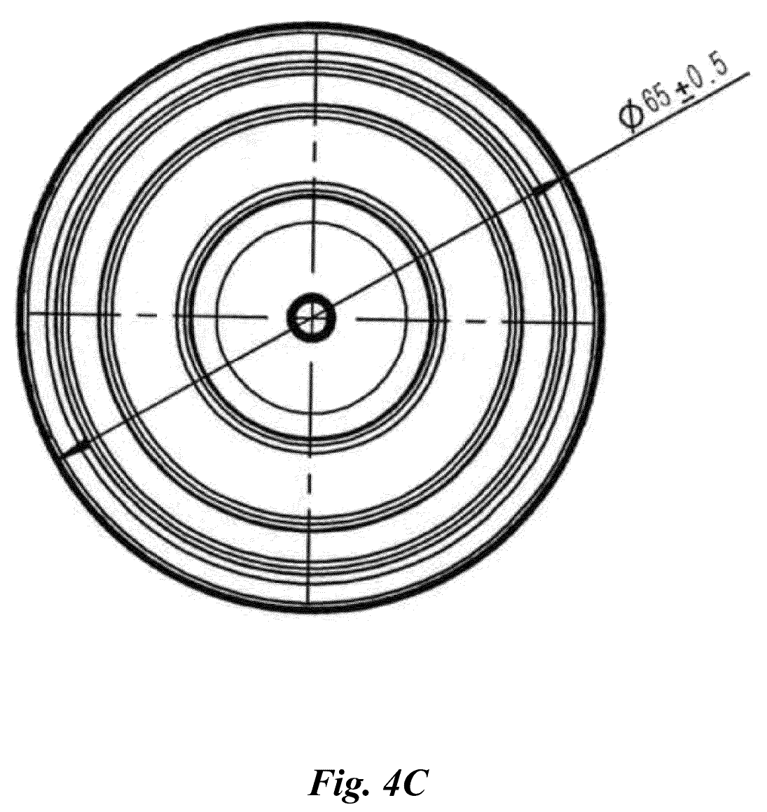

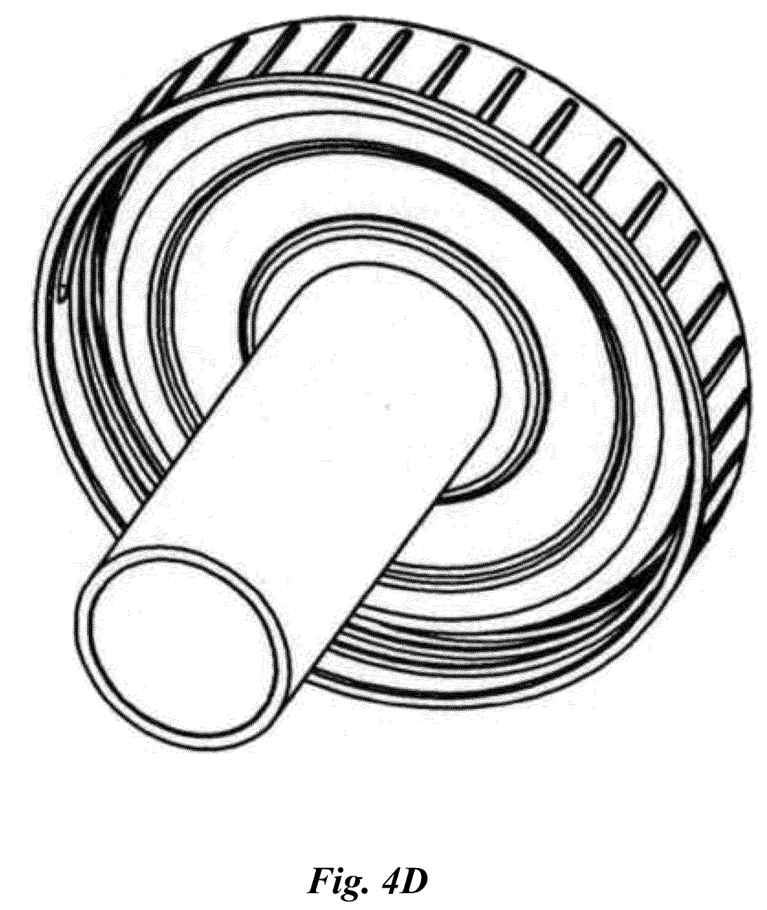

FIGS. 4A-4D show an example of a cap for use in a device for collection of a biological sample, including a side view of the cap (FIG. 4A), a cross-sectional side view of the cap inside a closed container (FIG. 4B), a top view of the cap (FIG. 4C), and an oblique view of the cap (FIG. 4D).

FIG. 4A provides example dimension for an example cap. Dimension for the diameter of the circular top portion of the cap, the diameter of the bottom portion that protrudes in to the container, the height of the top portion, the total height of the cap including the bottom portion that protrudes into the container, and a height of the lip of the cap are all shown.

FIG. 4B provides example dimension for the example cap. Dimension for the interior diameter of the circular top portion of the cap, the diameter of the interior of the bottom portion that protrudes in to the container, height of the bottom portion that protrudes into the container, and a diameter of the interior portions of the cap that make contact with the container (e.g., from the thread of the cap) are all shown. Additionally, the volume of the bottom portion of the cap which is able to hold a pierceable container can be at least 10 mL.

FIG. 4C shows the top view of the cap, with dimensions indicated for the diameter of the cap.

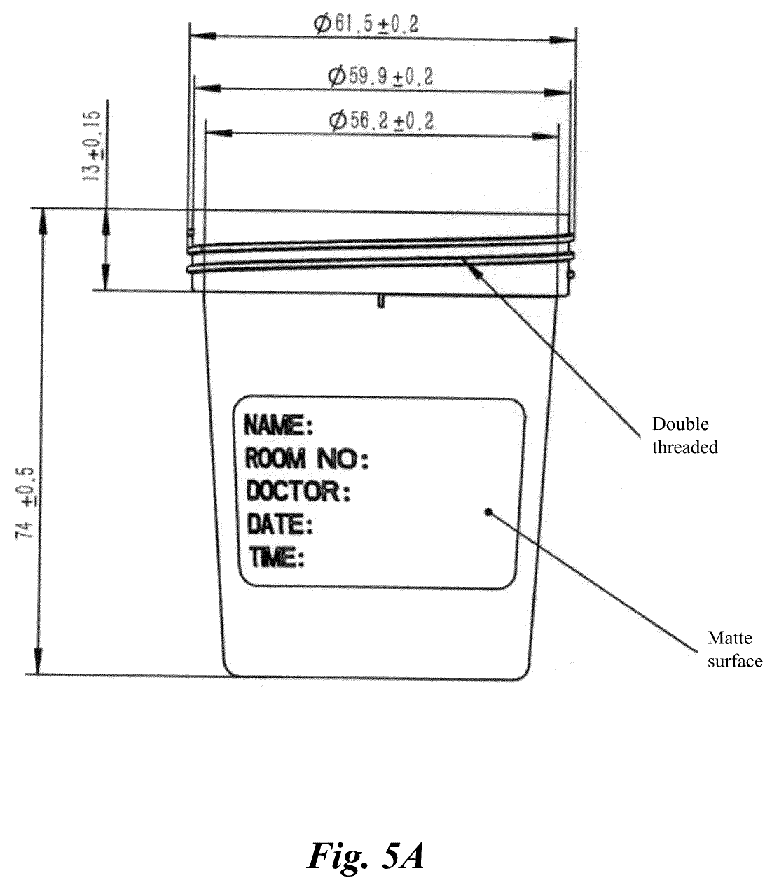

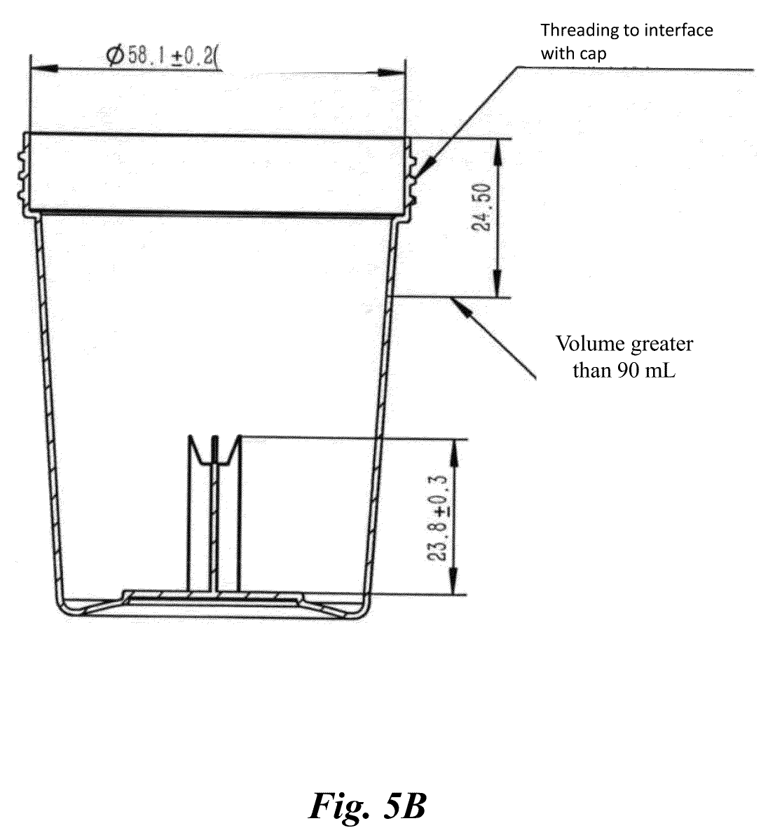

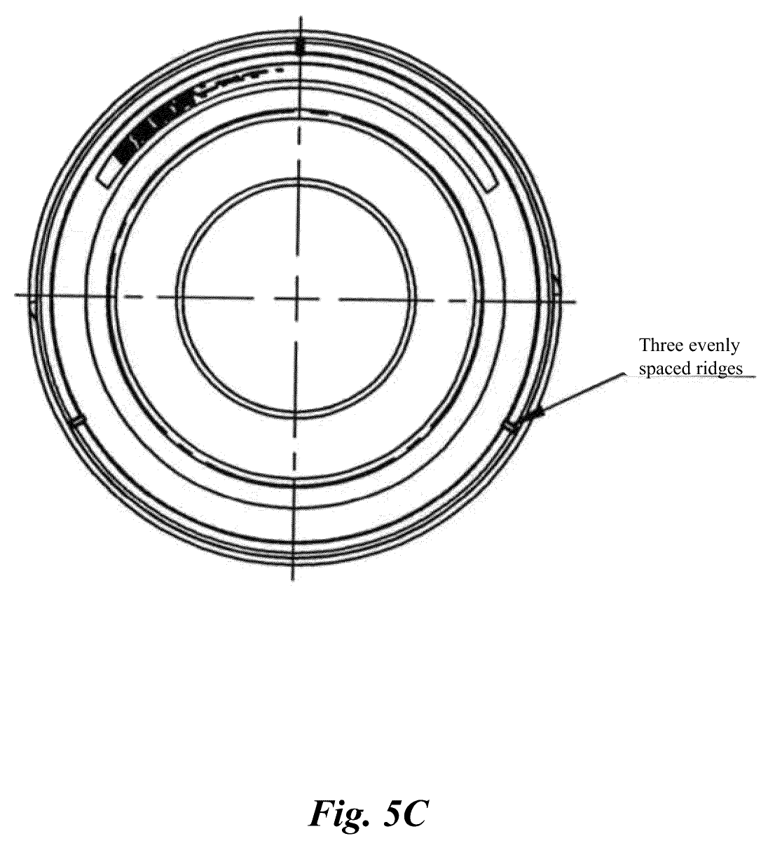

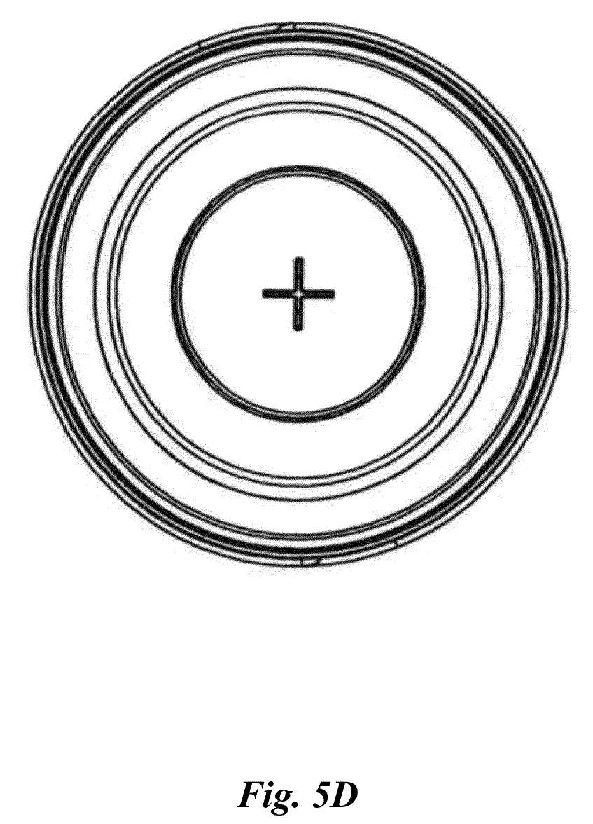

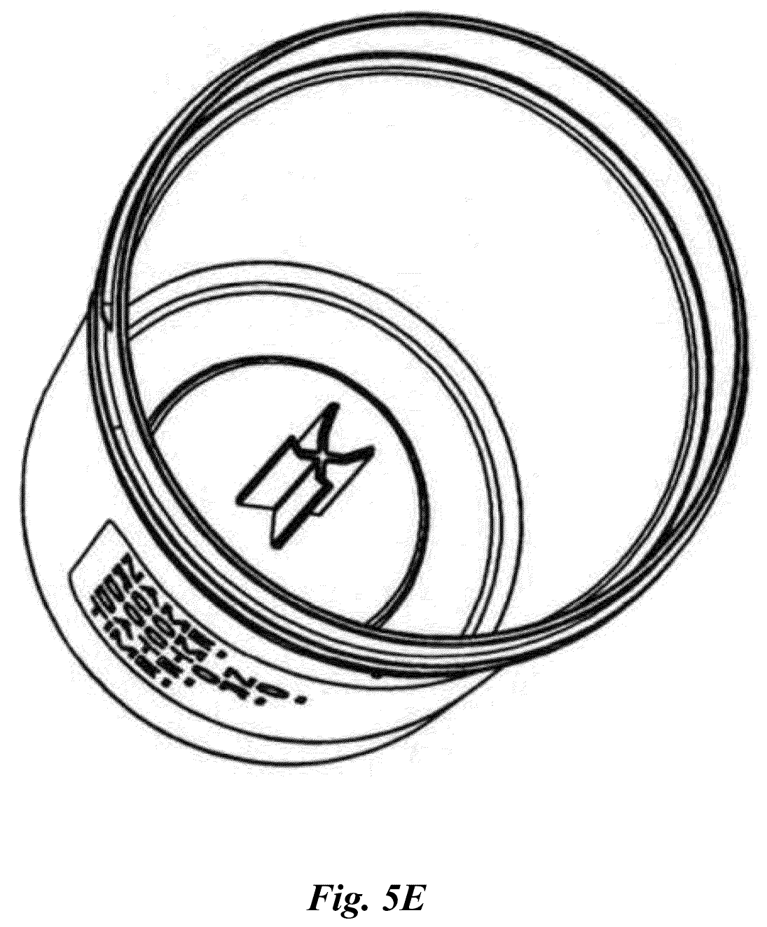

FIGS. 5A-5E show an example of a container for use in a device for collection of a biological sample, including a side view (FIG. 5A), a cross-sectional side view (FIG. 5B), a bottom view (FIG. 5C), a top view (FIG. 5D), and an oblique view (FIG. 5E).

FIG. 5A shows example dimension for interior diameter of the container, exterior diameter of the container from including the threading, exterior diameter of the container excluding the threading, height of the container, and the height of the threaded portion of the container which may contact the cap. The threads for contacting and screwing on the cap can be double threaded. This can allow for a strong contact with the cap to prevent the cap from inadvertently being removed. The container can also have an area with a matte surface. This matte surface can be written on with a writing implement (e.g., pen, pencil, etc.) to provide information about the sample by the subject or other individual.

FIG. 5B shows a cross-sectional view of the container with dimensions of the diameter of the interior of the container, the height of a portion of the cup with the threads, and the height of the piercing rod/piercer. The dimensions of the container may be such that the cap can interface snugly with the threads of the cap. Specifically, the threads of the container are designed to specifically interface with the threading on the cap, such to minimize any slippage of the cap from the container. The example container shown in FIG. 5B can hold a volume of greater than 90 mL.

FIG. 5C shows a bottom view of the example container. As shown, the bottom may have a three evenly spaced ridges. This may allow the container to rest evenly on a surface or interface with an instrument.

FIG. 5D shows a top view of the example container. The piercer/piercing rod can be in a “x” shape.

FIG. 5E shows an oblique view of the example container. As shown, the piercer/piercing rod can have the upraised points at the ends of the “x, with the middle of the “x” at a slightly lower height.

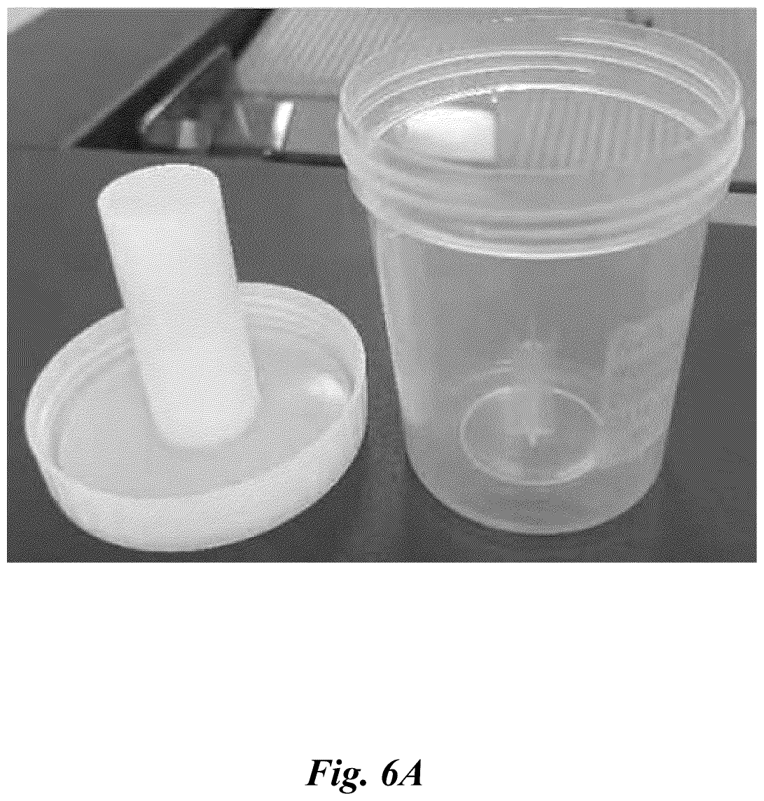

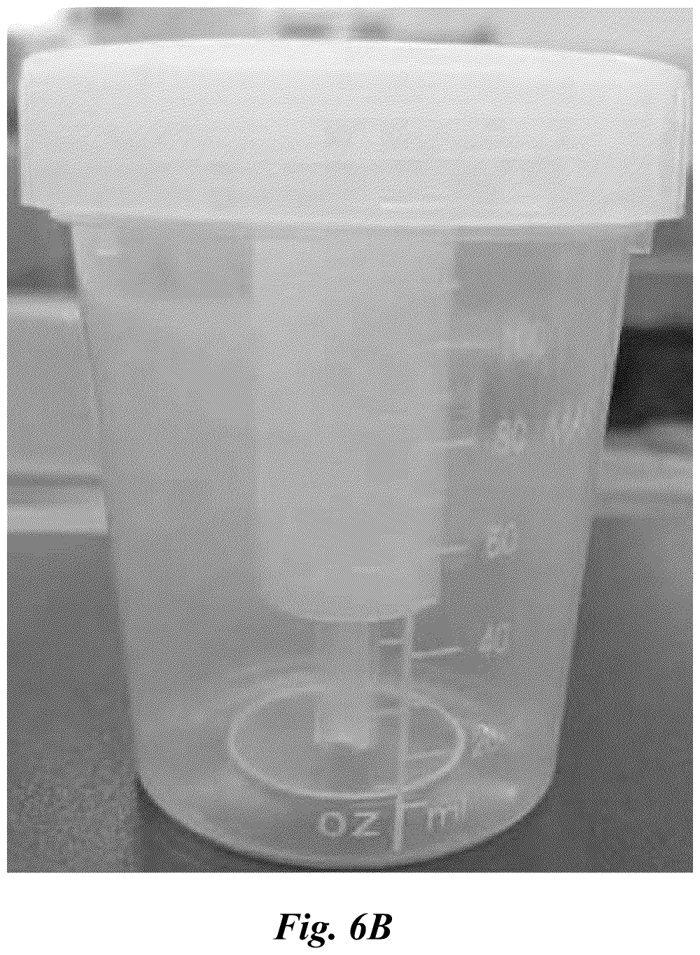

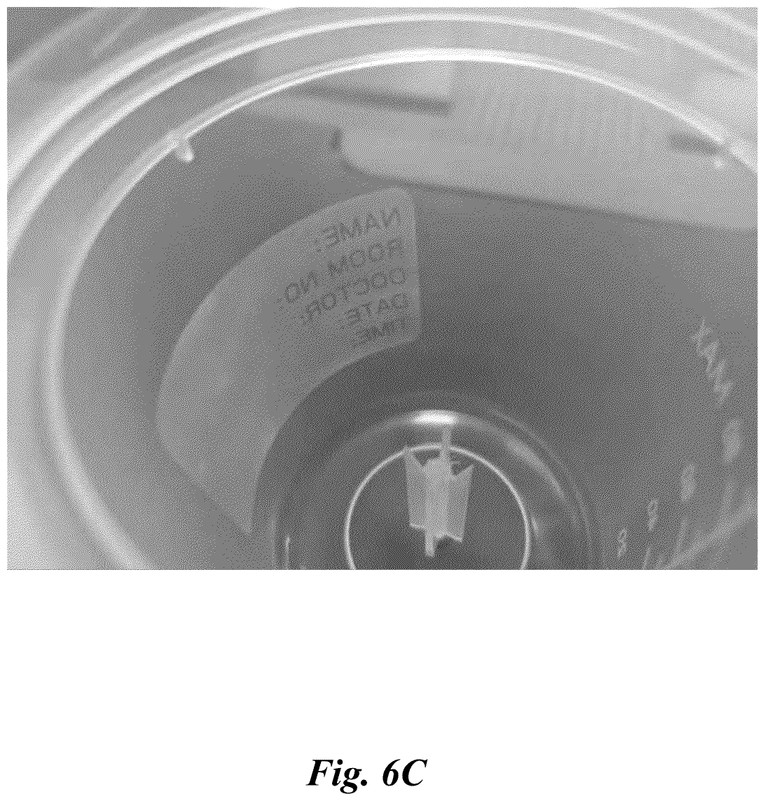

FIGS. 6A-6C show an example of a device for collection of a biological sample, including a view of an open configuration (FIG. 6A), a view of a closed configuration (FIG. 6B), and a top view of a container in an open configuration (FIG. 6C). FIGS. 6A-6C show photographs of an example device based on the schematics shown in FIGS. 3A-3C, 4A-4C, and 5A-5E.

In some embodiments, a sample from subject may be obtained or collected. The subject may be suffering from a cancer or suspected of having a cancer. The cancer may be specific or originating from an organ or other area of the subject. For example, the cancer may comprise breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. The cancer may comprise a hormone sensitive prostate cancer (HSPC), castrate-resistant prostate cancer (CRPC), metastatic prostate cancer, and a combination thereof.

The urine sample may comprise nucleic acids. The urine sample may be processed to obtain a cell-free deoxyribonucleic acid (cfDNA) sample or a cell-free ribonucleic acid (cfRNA) sample. The biological sample may comprise genomic DNA or germline DNA (gDNA). The nucleic acid may be a DNA (e.g. double-stranded DNA, single-stranded DNA, single-stranded DNA hairpins, cDNA, genomic DNA, germline DNA, circulating tumor DNA (ctDNA), cell-free DNA (cfDNA)), an RNA (e.g. cfRNA, mRNA, cRNA, miRNA, siRNA, miRNA, snoRNA, piRNA, tiRNA, snRNA), or DNA/RNA hybrids. In some case, the samples may comprise RNA and DNA. For example, a sample may comprise cfDNA and cfRNA, and the cfDNA and cfRNA may be analyzed. The urine sample may comprise polypeptides, lipids or carbohydrates. The polypeptides, lipids, or carbohydrates may be preserved or isolated for further analysis. The polypeptides, lipids, or carbohydrates may be degraded or denatured. such that the sample is enriched for nucleic acids.

In some embodiments, the pierceable container comprises one or more reagents. The reagents may stabilize the nucleic acid molecules, analytes, tissues, or cells in the sample. The reagents may minimize degradation of the biological sample prior to assaying. The additional reagents may comprise enzymes, enzymatic inhibitors, buffer salts, or chelators. For example, the reagents may comprise ethylenediaminetetraacetic acid (EDTA) or another chemical. The reagents may comprise a nuclease inhibitor.

The biological samples may be subjected to additional reactions or conditions prior to assaying. For example, the biological sample may be subjected to conditions that are sufficient to isolate, enrich, extract, deplete, or amplify nucleic acids, such cfDNA molecules or cfRNA molecules.

The methods disclosed herein may comprise conducting one or more enrichment reactions on one or more nucleic acid molecules in a sample. The use of the devices and methods provided herein may preserve or prevent degradation of nucleic acids and allow for additional nucleic acids compared to sample that were not subjected to the devices and methods provided herein. As such the output of the enrichment reactions may be improved by uses of the devices and methods of the present disclosure. The enrichment reactions may comprise contacting a sample with one or more beads or bead sets. The enrichment reactions may comprise one or more hybridization reactions. For example, the enrichment reactions may comprise contacting a sample with one or more capture probes or bait molecules that hybridize to a nucleic acid molecule of the biological sample. The enrichment reaction may comprise differential amplification of a set of nucleic acid molecules. The enrichment reaction may enrich for a plurality of genetic loci or sequences corresponding to genetic loci.

The methods disclosed herein may comprise performing one or more isolation or purification reactions on one or more nucleic acid molecules in a sample. The use of the devices and methods provided herein may preserve or prevent degradation of nucleic acids and allow for additional nucleic acids compared to sample that were not subjected to the devices and methods provided herein. As such the output of the isolation or purification reactions may be improved by use of the devices and methods of the present disclosure. The isolation or purification reactions may comprise contacting a sample with one or more beads or bead sets. The isolation or purification reaction may comprise one or more hybridization reactions, enrichment reactions, amplification reactions, sequencing reactions, or a combination thereof. The isolation or purification reaction may comprise the use of one or more separators. The one or more separators may comprise a magnetic separator. The isolation or purification reaction may comprise separating bead bound nucleic acid molecules from bead free nucleic acid molecules. The isolation or purification reaction may comprise separating capture probe hybridized nucleic acid molecules from capture probe free nucleic acid molecules. The isolation reactions may comprises removing or separating a group of nucleic acid molecules from another group of nucleic acids.

The methods disclosed herein may comprise conduction extraction reactions on one or more nucleic acids in a biological sample. The extraction reactions may lyse cells or disrupt nucleic acid interactions with the cell such that the nucleic acids may be isolated, purified, enriched or subjected to other reactions. The use of the devices and methods provided herein may preserve or prevent degradation of nucleic acids and allow for additional nucleic acids compared to sample that were not subjected to the devices and methods provided herein. As such the output of the extraction reactions may be improved by use of the devices and methods of the present disclosure.

The methods disclosed herein may comprise amplification or extension reactions on the sample or nucleic acids derived from the sample. The amplification reactions may comprise polymerase chain reaction (PCR). The amplification reaction may comprise PCR-based amplifications, non-PCR based amplifications, or a combination thereof. The one or more PCR-based amplifications may comprise PCR, quantitative PCR (qPCR), nested PCR, linear amplification, or a combination thereof. The one or more non-PCR based amplifications may comprise multiple displacement amplification (MDA), transcription-mediated amplification (TMA), nucleic acid sequence-based amplification (NASBA), strand displacement amplification (SDA), real-time SDA, rolling circle amplification, circle-to-circle amplification or a combination thereof. The amplification reactions may comprise an isothermal amplification. The use of the devices and methods provided herein may preserve or prevent degradation of nucleic acids and allow for additional nucleic acids compared to sample that were not subjected to the devices and methods provided herein. As such the output of the amplification or extension reactions may be improved by use of the devices or methods of this disclosure. The methods disclosed herein may comprise mass spectrometry measurements on the sample or analytes (e.g., DNA, RNA, proteins, and/or metabolites) derived from the sample.

In some embodiments, the nucleic acids may be subjected to sequencing reactions. The sequencing the reactions may be used on DNA, RNA or other nucleic acid molecules. Example of a sequencing reaction that may be used include capillary sequencing, next generation sequencing, Sanger sequencing, sequencing by synthesis, single molecule nanopore sequencing, sequencing by ligation, sequencing by hybridization, sequencing by nanopore current restriction, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing, pH-mediated sequencing, semiconductor sequencing or a combination thereof. The sequencing reactions may comprise whole genome sequencing, whole exome sequencing, low-pass whole genome sequencing, targeted sequencing, methylation-aware sequencing, enzymatic methylation sequencing, bisulfite methylation sequencing. The sequencing reaction may be a transcriptome sequencing, mRNA-seq, totalRNA-seq, smallRNA-seq, exosome sequencing, or combinations thereof. Combinations of sequencing reactions may be used in the methods provided herein. For example, a sample may be subjected to whole genome sequencing and whole transcriptome sequencing. As the samples may comprise multiple types of nucleic acids (e.g. RNA and DNA), sequencing reactions specific to DNA or RNA may be used such to obtain sequence reads relating to the nucleic acid type.

The sequencing of nucleic acids may generate sequencing read data. The sequencing reads may be processed such to generate data of improved quality. The sequencing reads may be generated with a quality score. The quality score may indicate an accuracy of a sequence read or a level or signal above a nose threshold for a given base call. The quality scores may be used for filtering sequencing reads. For example, sequencing reads may be removed that do not meet a particular quality score threshold. The sequencing reads may be processed such to generate a consensus sequence or consensus base call. A given nucleic acid (or nucleic acid fragment) may be sequenced and errors in the sequence may be generated due to reactions prior or during sequencing. For example, amplification or PCR may generate error in amplicons such that the sequences are not identical to a parent sequence. Using sample barcodes or molecular barcodes, error correction may be performed. Error correction may include identifying sequence reads that do not corroborate with other sequences from a same sample or same original parent molecules. The use of barcodes may allow the identification or a same parent or sample. Additionally, the sequence reads may be processed by performing single strand consensus calling or double stranded consensus call, thereby reducing or suppressing error.

In some embodiments, the devices and methods of the present disclosure may allow an improvement of an accuracy, sensitivity, or specificity of detection of the presence of molecule in the sample, as compared to a sample that is not preserved or stabilized using the methods or devices disclosed herein. For example, the devices and methods may comprise improving detecting of the presence of molecule in the sample in the subject at an accuracy of at least about 60%, at least about 70%, at least about 75%, at least about 80%, at least about 85%, at least about 90%, at least about 95%, at least about 98%, or at least about 99%, as compared to a sample that is not preserved or stabilized using the methods or devices disclosed herein. The devices and methods may comprise detecting of the presence of molecule in the sample in the subject at a sensitivity of at least about 60%, at least about 70%, at least about 75%, at least about 80%, at least about 85%, at least about 90%, at least about 95%, at least about 98%, or at least about 99%, as compared to a sample that is not preserved or stabilized using the methods or devices disclosed herein. The devices and methods may comprise detecting of the presence of molecule in the sample in the subject at a specificity of at least about 60%, at least about 70%, at least about 75%, at least about 80%, at least about 85%, at least about 90%, at least about 95%, at least about 98%, or at least about 99%, as compared to a sample that is not preserved or stabilized using the methods or devices disclosed herein.

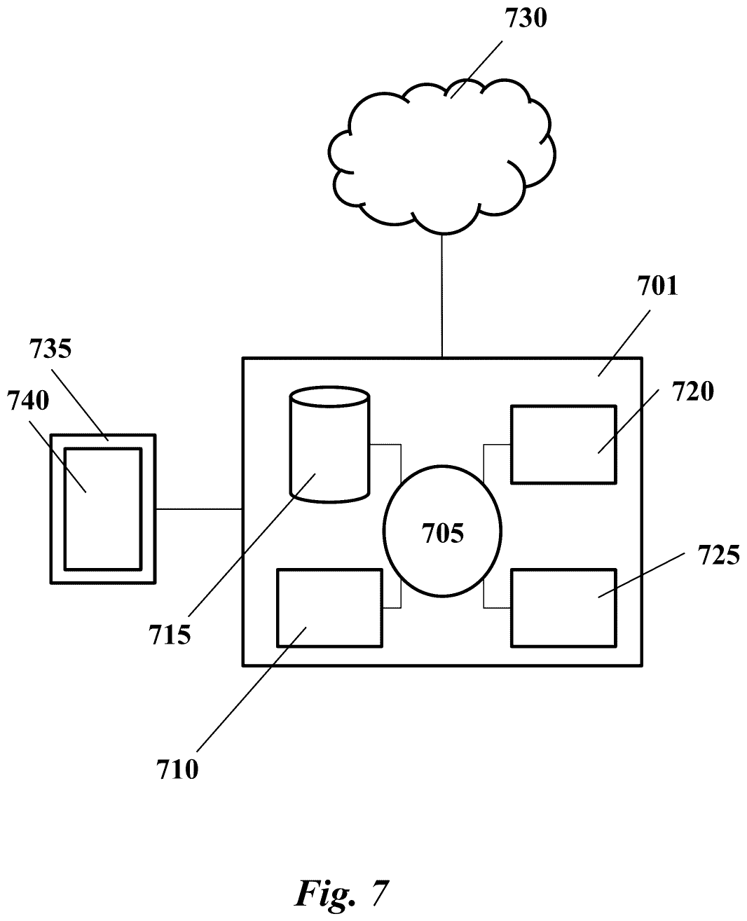

The present disclosure provides computer systems that are programmed to implement methods of the disclosure. FIG. 7 shows a computer system 701 that is programmed or otherwise configured to, for example, collect a biological sample of a subject.

The computer system 701 can regulate various aspects of analysis, calculation, and generation of the present disclosure, such as, for example, collecting a biological sample of a subject. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet.

The network 730 in some cases is a telecommunication and/or data network. The network 730 can include one or more computer servers, which can enable distributed computing, such as cloud computing. For example, one or more computer servers may enable cloud computing over the network 730 (“the cloud”) to perform various aspects of analysis, calculation, and generation of the present disclosure, such as, for example, collecting a biological sample of a subject. Such cloud computing may be provided by cloud computing platforms such as, for example, Amazon Web Services (AWS), Microsoft Azure, Google Cloud Platform, and IBM cloud. The network 730, in some cases with the aid of the computer system 701, can implement a peer-to-peer network, which may enable devices coupled to the computer system 701 to behave as a client or a server.

The CPU 705 may comprise one or more computer processors and/or one or more graphics processing units (GPUs). The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for performing methods such as, for example, collecting a biological sample of a subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, collect a biological sample of a subject.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-60. (canceled)

61. A device for collecting a biological sample of a subject, comprising:
a container comprising a piercer extending across a longitudinal axis of said container towards an opening of said container; and
a cap comprising a pierceable container comprising a reagent for processing or preserving said biological sample,
wherein said device is configured such that upon engagement of said cap with said container to dispose said cap adjacent to said opening, said piercer pierces said pierceable container to release said reagent into said container.

62. The device of claim 61, wherein said piercer pierces said pierceable container upon rotation of said cap or said container.

63. The device of claim 61, wherein said engagement of said cap with said container to dispose said cap adjacent to said opening, and movement of said cap towards said container or movement of said container towards said cap, closes said container.

64. The device of claim 61, wherein said cap is configured to be attached to said container at least in part by exerting a pressure on said cap towards said container or exerting a pressure on said container towards said cap, when said cap is adjacent to said opening.

65. The device of claim 61, wherein said cap is configured to be attached to said container at least in part by rotating said cap or said container, when said cap is adjacent to said opening.

66. The device of claim 61, wherein said attaching of said cap to said container generates a leak-resistant, leak-proof, or airtight compartment.

67. The device of claim 61, wherein said cap and said container each comprises threading, wherein said threading of said cap and said threading of said container are configured to interface together thereby closing said container.

68. The device of claim 61, wherein said piercer comprises a rod, wherein a top of said rod comprises a sharp protrusion.

69. The device of claim 61, wherein said pierceable container is configured such that a pierceable surface of said pierceable container is exposed on a bottom surface of said cap.

70. The device of claim 61, wherein said reagent is configured to preserve integrity of analytes in said biological sample, responsive to said releasing of said reagent into said container.

71. The device of claim 70, wherein said analytes comprise nucleic acids, polypeptides, lipids, or carbohydrates.

72. The device of claim 61, wherein said container comprises at least one of: a tube, a flat bottom or a substantially flat bottom, and a round bottom or a substantially round bottom.

73. The device of claim 61, wherein said reagent comprises an enzyme, an enzyme inhibitor, a chelator, a buffering agent, or a combination thereof.

74. The device of claim 61, wherein said biological sample comprises a urine sample.

75. A method for collecting a biological sample of a subject, comprising:
(a) providing a container comprising a piercer extending across a longitudinal axis of said container towards an opening of said container, wherein said container comprises said biological sample;
(b) engaging a cap with said container to dispose said cap adjacent to said opening, wherein said cap comprises a pierceable container comprising a reagent for processing or preserving said biological sample; and
(c) upon engaging said cap with said container, subjecting said cap or said container to a closing motion, thereby piercing, by said piercer, said pierceable container to release said reagent into said container.

76. The method of claim 75, wherein said closing motion comprises rotating said container or said cap.

77. The method of claim 75, wherein said closing motion comprises exerting a pressure on said cap towards said container or exerting a pressure on said container towards said cap.

78. The method of claim 75, further comprising closing said container at least in part by rotating said cap or said container.

79. The method of claim 75, further comprising closing said container at least in part by exerting a pressure on said cap or said container.

80. The method of claim 75, further comprising attaching said cap to said container thereby generating a leak-resistant, leak-proof, or airtight compartment.

81. The method of claim 75, wherein said cap and said container each comprise threading, and wherein said method further comprises interfacing together said threading of said cap and said threading of said container, thereby closing said container.

82. The method of claim 75, wherein said piercer comprises a rod, wherein a top of said rod comprises a sharp protrusion.

83. The method of claim 75, wherein said pierceable container is configured such that a pierceable surface of said pierceable container is exposed on a bottom surface of said cap.

84. The method of claim 75, further comprising mixing together said reagent and said biological sample, responsive to said releasing of said reagent into said container.

85. The method of claim 84, further comprising preserving, using said reagent, integrity of analytes in said biological sample, responsive to said mixing.

86. The method of claim 85, wherein said analytes comprise nucleic acids, polypeptides, lipids, or carbohydrates.

87. The method of claim 85, further comprising assaying said preserved analytes.

88. The method of claim 75, wherein said container comprises at least one of: a tube, a flat bottom or a substantially flat bottom, and a round bottom or a substantially round bottom.

89. The method of claim 75, wherein said reagent comprises an enzyme, an enzyme inhibitor, a chelator, a buffering agent, or a combination thereof.

90. The method of claim 75, further comprising providing said biological sample to said container.
